(19) United States
(12) Patent Application Publication
(10) Pub. No.: US 2010/0274014 A1
(43) Pub. Date: Oct. 28, 2010

(54) PROCESS FOR MANUFACTURING ROSUVASTATIN POTASSIUM AND CRYSTALLINE AND AMORPHOUS FORMS THEREOF

(75) Inventors: Dhimant Jasubhai Patel, Gujarat (IN); Rajiv Kumar, Gujarat (IN); Virendra Kumar Agarwal, Gujarat (IN)

Correspondence Address: LADAS & PARRY LLP, 26 WEST 61ST STREET, NEW YORK, NY, 10023, US

(73) Assignee: CADILA HEALTHCARE LIMITED, Gujarat (IN)

(21) Appl. No.: 12/161,455

(22) PCT Filed: Jan. 25, 2007
(86) PCT No.: PCT/IN2007/000037

§ 371 Date: Jul. 9, 2010

(30) Foreign Application Priority Data
Jan. 30, 2006 (IN) 1217/MUM/2005

(51) Int. Cl. C07D 239/42

(52) U.S. Cl. 544/297

(57) ABSTRACT

A process of manufacturing of Rosuvastatin Potassium of formula (I)

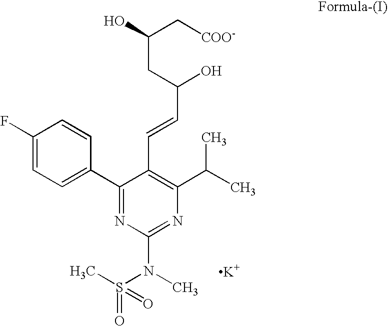

is disclosed. The process comprises the steps of
(a) treating Rosuvastatin protected compound of formula (II)

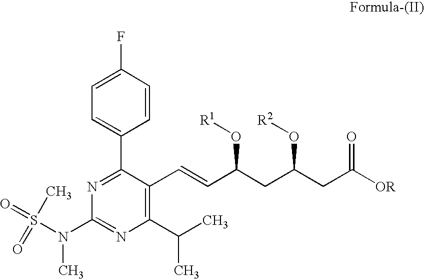

wherein R¹ and R² are same or different having C₁-C₄ carbon atom or hydrogen or R¹ and R² can combine together to afford a cyclic structure comprising a junction atom as carbon or metal atom such as Si (silicon);
with an inorganic base of the kind such as herein described having potassium as cation in a suitable solvent to form Rosuvastatin potassium;
(b) isolating Rosuvastatin potassium.

### FIELD OF THE INVENTION

The present invention relates to a process for the manufacture of potassium salt of (E) -7-[4-(4-fluorophenyl)-6-isopropyl-2-[methyl (methylsulfonyl)amino]5-pyrimidin-5-yl](3R,5S)-3,5-dihydroxy-6-heptenoic acid

### BACKGROUND OF THE INVENTION

US RE 37314 (Reissue of U.S. Pat. No. 5,260,440) discloses Rosuvastatin that is chemically known as (E)-7-[4-(4-fluorophenyl)-6-isopropyl-2-[methyl (methylsulfonyl)amino]pyrimidin-5-yl](3R,5S)-3,5-dihydroxy-6-heptenoic acid and its salts, which are HMG CoA reductase inhibitors and useful in the treatment of hypercholesterolemia, hyperlipoproteinemia and atherosclerosis.

The '314 patent discloses the existence of Rosuvastatin in the generic formula and its various alkali metal salts i.e., lithium, sodium, potassium, and cesium, as well as alkaline earth metal salts are beryllium, magnesium, and calcium. However '314 patent is limited in its disclosure to an amorphous (powder) form of the calcium salt of Rosuvastatin, which is prepared by isolating its precursor sodium salt. State of the sodium salt obtained in '314 patent is defined as “powdery crystals”.

A powdery or amorphous form of a compound intended for pharmaceutical use may give rise to manufacturing problems and there is therefore a need to identify alternative salt of rosuvastatin that is crystalline salt. Such crystalline salt can generally be purified more easily than an amorphous form and may possess other advantageous properties, for example in relation to their particular crystalline form and/or their solubility characteristics and/or their lack of hygroscopicity and/or their stability characteristics, including their thermal stability properties and/or their ability to undergo oxidative degradation.

WO2005068435 discloses a method of preparation of the amorphous hemi-calcium salt of rosuvastatin by a one-pot manufacturing process from the Rosuvastatin ester or lactone intermediate. The invention describes use of alkali metal hydroxides for the purpose of the hydrolysis of Rosuvastatin ester or lactone intermediate in a suitable solvent system, which is subjected to the treatment of Calcium acetate or Calcium hydroxide to afford amorphous hemicalcium salt of Rosuvastatin without isolating any intermediate alkali metal salt of Rosuvastatin.

WO 2005077917 describes the process for preparation of novel amorphous rosuvastatin magnesium from crystalline rosuvastatin magnesium, rosuvastatin methyl ammonium salt and from Rosuvastatin lactone. In this patent use of potassium hydroxide, potassium carbonate or potassium bicarbonates disclosed for the purpose of the hydrolysis of Rosuvastatin lactone but use of the bases containing cation potassium are not exemplified in the invention. Also any intermediate step having alkali metal salt of Rosuvastatin is not isolated.

WO2004/014872 discloses an improved process for manufacturing rosuvastatin calcium salt. According to this patent publication, various ammonium salts of Rosuvastatin is subjected to the treatment of inorganic bases containing alkali metal cations. The in-situ obtained Rosuvastatin alkali metal salt is converted to its corresponding calcium salt by means of reacting Rosuvastatin alkali metal salt with calcium chloride dihydrate. The isolation of potassium salt is not exemplified in this patent.

WO2004/108691 discloses an improved process for manufacturing calcium salt of rosuvastatin, in this patent various alkali metal hydroxide have used for the hydrolysis of Rosuvastatin ester in a suitable aqueous solvent system. However use of potassium hydroxide for the purpose of hydrolysis or isolation of potassium salt is not exemplified within the art.

U.S. Pat. No. 6,841,554 discloses various crystalline ammonium, lithium and magnesium salts of rosuvastatin. Even though ammonium salt of rosuvastatin are not likely to be used for administration to a patient, this patent only teaches a method for purifying rosuvastatin through crystallization.

U.S. Pat. No. 6,589,959 disclose the process for preparation of crystalline form of rosuvastatin calcium salt (Form-A) by warming the amorphous form of rosuvastatin calcium.

WO 2005051921 has described the alkyl ammonium crystalline salts of rosuvastatin that provide for purification of rosuvastatin and its pharmaceutically acceptable salts also claiming the XRPD peak values.

In all the prior art purpose of the invention is related to the isolation of amorphous Rosuvastatin calcium salt that involves in-situ formation of various alkali metal salts of Rosuvastatin, which are not isolated. Moreover various prior art teaches obtaining crystalline salts of Rosuvastatin but not the method to isolate the Rosuvastatin potassium salt or its purification.

Various prior arts describe amorphous salts of Rosuvastatin. The amorphous form has its advantages and disadvantages that it is not suitable from commercial point of view because, the amorphous product is difficult to isolate and the product is not obtained in high purity. Moreover, it is difficult while handling amorphous product at various unit operation stages because of the problem of dusting, hence handling amorphous products requires installing special equipments to overcome health hazards.

However, amorphous form has its advantages such as high surface area that helps increasing solubility profile of the drug substance and in some cases different bio-availability pattern compared to the crystalline form (Konne T., Chem. Pharm. Bull., 38 2003 (1990)).

There is therefore, a need for a rosuvastatin salt with improved pharmaceutical characteristics, also the present invention alleviates the hitherto problems associated with prior art rosuvastatin salts as described above.

Prior art also provides a basis for the present invention because the potassium salt has not isolated within the art though potassium hydroxide is disclosed for the use in saponification for obtaining rosuvastatin.

### SUMMARY OF THE INVENTION

The present invention provides a process of manufacturing of Rosuvastatin Potassium of formula (I) comprising the steps of

(a) treating Rosuvastatin protected compound of formula (II)

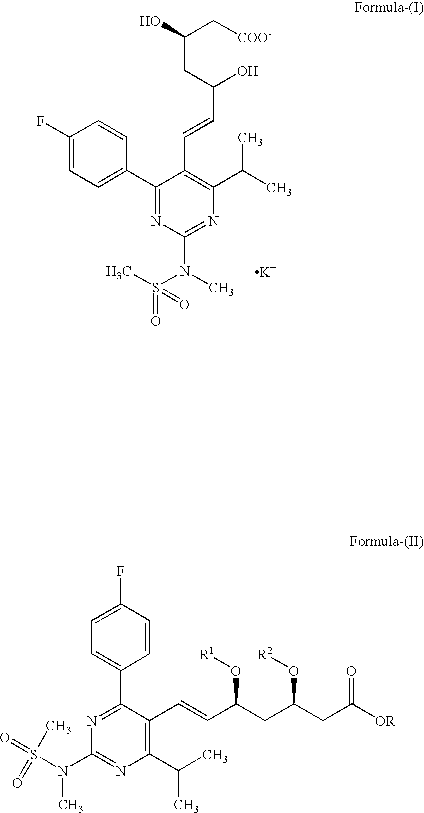

wherein R¹ and R² are same or different having C₁-C₄ carbon atom or hydrogen or R¹ and R² can combine together to afford a cyclic structure comprising a junction atom as carbon or metal atom such as Si (silicon);
with an inorganic base of the kind such as herein described having potassium as cation in a suitable solvent to form Rosuvastatin potassium;
(b) isolating Rosuvastatin potassium.

In a preferred feature, said inorganic base is selected from potassium hydroxide, potassium bicarbonate, potassium carbonate, potassium tert-butoxide, potassium alcoholate

In another preferred feature, the mole ratio of said Rosuvastatin protected compound of formula (II) to said inorganic base 1:1.25.

In another preferred feature, said solution is further cooled to 0° C. to 10° C. preferably to 5° C. to 10° C. and potassium hydroxide is added to give Rosuvastatin potassium salt.

In another preferred feature, the concentration of the solution is further reduced to by unit operation distillation, whereby maximum solvent is removed under vacuum at 50 to 55° C. temperature.

In another preferred feature, said solvent is an alcohol selected front the group consisting of methanol, ethanol, isopropanol, n-butanol, isobutanol or mixtures thereof, preferably methanol.

In another preferred feature, said Rosuvastatin potassium is isolated in an amorphous form.

In another preferred feature, said Rosuvastatin potassium is further crystallized from suitable solvent to obtain crystalline Rosuvastatin potassium.

In another preferred feature, said suitable solvent is acetonitrile.

The present invention also relates to crystalline and amorphous Rosuvastatin potassium prepared in accordance with the present invention . . . claim is in isolated form.

### BRIEF DESCRIPTION OF THE ACCOMPANYING DRAWINGS

In the accompanying drawings:

FIG. 1: shows the X-Ray diffraction pattern of amorphous Rosuvastatin Pottassium of the present invention;

FIG. 2: shows the X-Ray diffraction pattern of crystalline Rosuvastatin Pottassium of the present invention;

### DETAILED DESCRIPTION

According to the present invention there is provided a potassium salt of the compound (E)-7-[4-(4-fluorophenyl)-6-isopropyl-2-[methyl(methylsulfonyl)amino]pyrimidin-5-yl](3R,5S)-3,5-dihydroxy-6-heptenoic acid in amorphous as well as crystalline form.

The Rosuvastatin potassium salt of the present invention is represented by the formula (I)

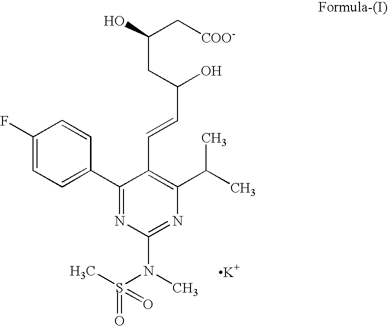

More particularly, the present invention provides a crystalline, amorphous and solvate form of rosuvastatin potassium salt, which can be well characterized by its unique X-ray powder diffraction pattern.

Derivatization is a part of the purification technique. Hence, it is also a preferred embodiment of present invention to utilize pure Rosuvastatin potassium salt, which is free from its enantiomeric as well as process related impurities and thus suitable for the preparation of crystalline or amorphous form of Rosuvastatin calcium salt.

Pure Rosuvastatin potassium salt can be derived from its various intermediate forms such as solvates preferably alcoholates and hydrates or from an amorphous

Rosuvastatin potassium salt.

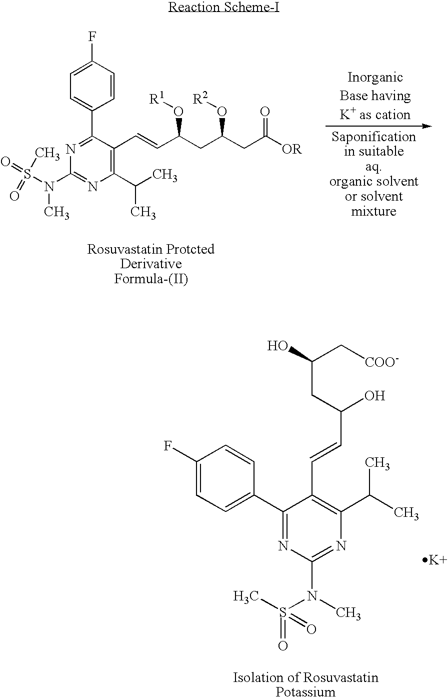

For the purpose of this invention Rosuvastatin potassium salt can be isolated by hydrolysis of the compounds of formula—(II) with the help of inorganic base having K⁺ as cation such as potassium hydroxide, potassium carbonate, potassium bicarbonate, potassium alcoholate etc. in a suitable aqueous organic solvent or solvent mixture.

If required for the purpose of de-protecting the Rosuvastatin protected diol ester intermediate first it may be subjected to the treatment of acidic hydrolysis in suitable aqueous organic solvent system.

For the purpose of this invention for preparing potassium salt of Rosuvastatin, the precursor compound of the Formula—(II) can be used, wherein R¹ and R² are sable or different having C₁-C₄ carbon atom or hydrogen. Both R¹ and R² can combine together to afford a cyclic structure comprising a junction atom as carbon or metal atom such as Si (silicon). Both R¹ and R² represent hydroxyl protecting groups.

Most preferably, for the purpose of this invention compound of the Formula—(II) is selected as methyl ester of Rosuvastatin, which can be obtained by the process as disclosed in U.S. Pat. No. 5,260,440.

As an end result of the hydrolysis process as described in the Reaction Scheme-I, technical grade of Rosuvastatin potassium may obtained, which can be further converted into pure Rosuvastatin potassium by involving purification steps comprising intermediate stages such a solvate of various solvents.

If desired solvates of Rosuvastatin potassium can intentionally be prepared, which may be subjected to dissolvation for obtaining pure form of Rosuvastatin potassium.

For the purpose of isolating pure Rosuvastatin potassium salt from the reaction medium, the resultant solution after the completion of the saponification reaction can be subjected to vacuum drying, lyophilization (freeze drying) or spray drying. As an end result of these process amorphous forms of Rosuvastatin potassium may be obtained, which can be converted to crystalline Rosuvastatin potassium.

As a preferred embodiment of this invention the Rosuvastatin potassium is useful as HMG CoA reductase inhibitor for treating hyperlipidemia, comprising administrating to a mammal in need there of a therapeutically effective amount.

Suitably, a Rosuvastatin potassium salt according to the present invention may be formulated for administration by any route, and examples are oral, rectal, topical, parental, intravenous or intramuscular administration. Preparations may it desired, be designated to give slow release of a Rosuvastatin potassium salt by exploiting specific nature of its form according to the present invention.

Yet another preferred embodiment of the invention is to use Rosuvastatin potassium, salt, which is pure from its process and enantiomeric impurities for the purpose of the preparation of Rosuvastatin calcium salt.

The invention is further illustrated, but not limited by following example.

### Examples 1

### Preparation of Amorphous Form of Rosuvastatin Potassium

In a 2 l four neck reaction flask equipped with a mechanical stirrer and temperature as well as pH monitoring facility, 625 ml of Methanol is added. To this reaction vessel 25.0 g Rosuvastatin protected diol of Formula—(IIa) is added under stirring. A solution thus obtained is cooled to 5-10° C., then the mixture of hydrochloric acid (7.5 ml) and water (52.5 ml) is added slowly within 20 minutes time. After complete addition, solution in the reaction flask is stirred it at 5-10° C. for 15 minutes. The resultant solution is warmed to 30-35° C. and stir for 45 minutes.

Reaction is monitored at this stage by Thin Layer Chromatography.

Again the stirred solution in the reaction vessel is cooled to 5-10° C. then slowly the solution of potassium hydroxide is added, which is made by dissolving 12.2 g of potassium in 122 ml of water at 5-10° C. The saponification is continued for 15 minutes under vigorous stirring at the same temperature. The temperature of the saponification reaction is increased up to 20-30° C. while continuing the stirring and the same condition is maintained for 30 minutes.

Reaction is monitored at this stage by Thin Layer Chromatography.

The resultant solution at the end of the saponification reaction is concentrated to be half of the volume by unit operation distillation at 50-55° C. under vacuum. Then clear solution is washed with 500 ml of toluene. Again it is subjected to distillation wherein maximum amount of solvent methanol is removed at 50-55° C. under vacuum. Traces of methanol are removed by adding 200 ml of isopropanol and azeotropic distillation is carried out under vacuum at 52° C. temperature. In same reaction flask again 150 ml of isopropanol is added that results separation of potassium chloride salt solid at 25-35° C. temperature and the suspension is filtered off. The potassium chloride is removed by filtration. The mother liquor obtained was distilled to get amorphous form of rosuvastatin potassium.

### Example: 2

### Preparation of Crystalline Form of Rosuvastatin Potassium

In the mother liquor obtained from Example—1, 50 ml of acetonitrile is added and resultant solution is concentrated by distillation under vacuum and there after it is allowed to cool. Potassium salt of rosuvastatin obtained by filtration is crystalline form.

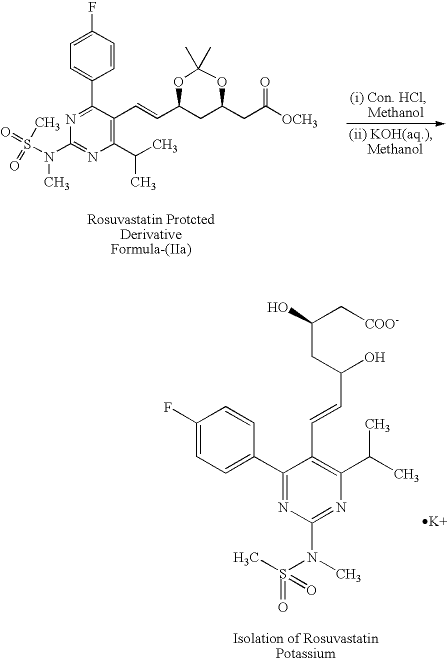

1. A process for manufacturing Rosuvastatin Potassium of formula (I) 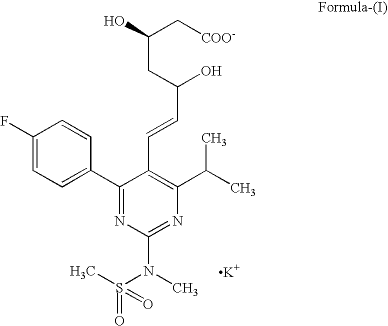
comprising the steps of
(a) treating Rosuvastatin protected compound of formula (II) 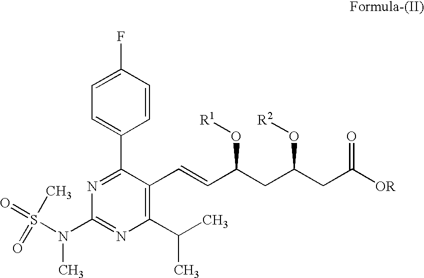
wherein R¹ and R² are same or different and are selected from C₁-C₄ carbon atoms and hydrogen or R¹ and R² together form a cyclic structure comprising a junction atom as carbon or metal atom such as Si (silicon); with an inorganic base having potassium as cation in a suitable solvent to form Rosuvastatin potassium; and
(b) isolating Rosuvastatin potassium.

2. A process as claimed in claim 1, wherein said inorganic base is selected from the group consisting of potassium hydroxide, potassium bicarbonate, potassium carbonate, potassium tert-butoxide, and potassium alcoholate.

3. The process as claimed in claim 1, wherein the mole ratio of said Rosuvastatin protected compound of formula (II) to said inorganic base 1:1.25.

4. The process as claimed in claim 1, wherein said solution is further cooled to 0° C. to 10° C. and potassium hydroxide is added to obtain Rosuvastatin potassium salt.

5. The process as claimed in claim 1, wherein the concentration of the solution is further reduced by unit operation distillation, whereby maximum solvent is removed under vacuum at 50 to 55° C. temperature.

6. The process as claimed in claim 1, wherein said solvent is an alcohol selected from the group consisting of methanol, ethanol, isopropanol, n-butanol, isobutanol and mixtures thereof.

7. The process as claimed in claim 1, wherein said Rosuvastatin potassium is isolated in an amorphous form.

8. The process as claimed in claim 1, wherein said Rosuvastatin potassium is further crystallized from suitable solvent to obtain crystalline Rosuvastatin potassium.

9. The process as claimed in claim 8, wherein said solvent is acetonitrile.

10. A crystalline Rosuvastatin potassium.

11. A crystalline Rosuvastatin potassium as claimed in claim 10, characterized by X-ray powder diffraction (XRD) having main peaks at 3.44, 6.74, 9.71, 10.09, 11.81, 16.86, 20.26, 21.53, 25.41, 26.83, 28.43, 34.31±0.2 degree two theta.

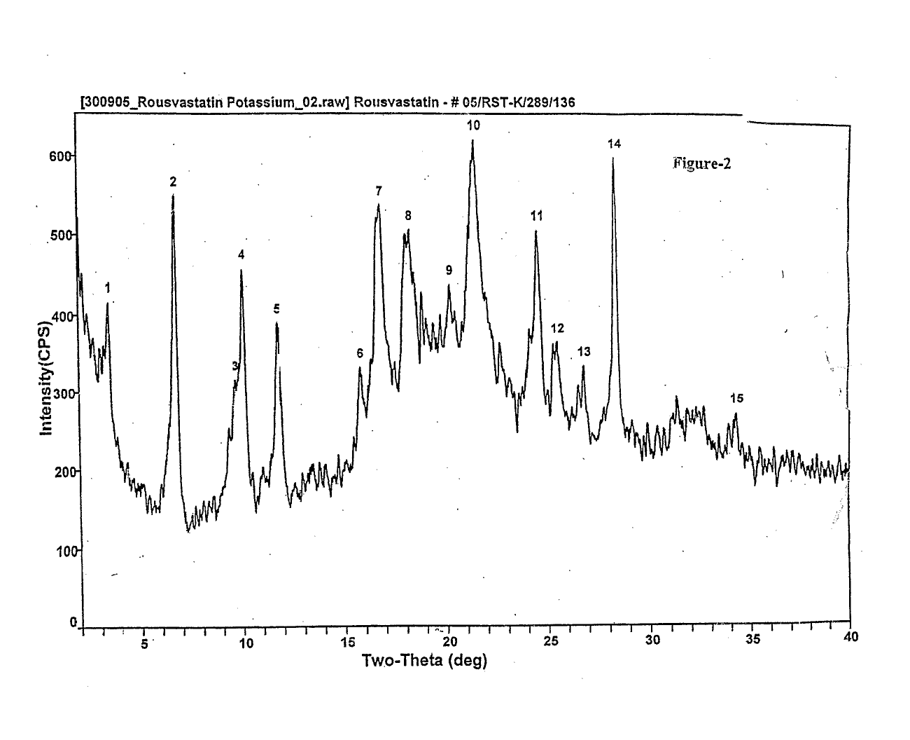

12. A crystalline Rosuvastatin Potassium as claimed in claim 11 having the X-Ray powder diffraction pattern as shown in FIG. 2.

13. An amorphous Rosuvastatin potassium.

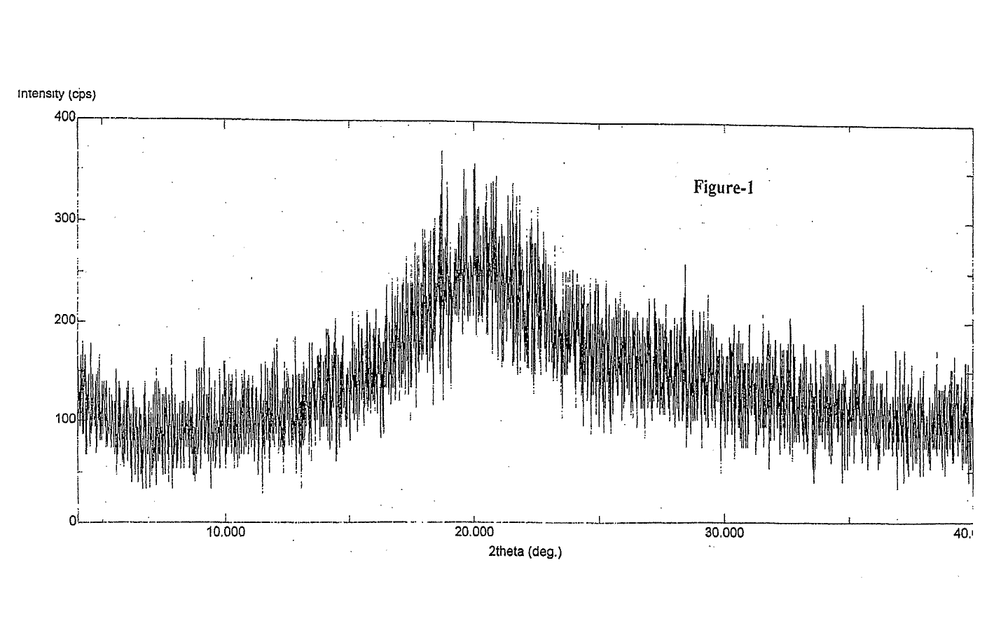

14. An amorphous Rosuvastatin Potassium as claimed in claim 13 having the X-Ray powder diffraction as shown in FIG. 1.

15. (canceled)

16. The process according to claim 4, wherein said solution is cooled to 0° C. to 10° C.

17. The process according to claim 6, wherein the alcohol is methanol.
